(19) United States
(12) Patent Application Publication
(10) Pub. No.: US 2020/0363624 A1
(43) Pub. Date: Nov. 19, 2020

(54) OPTICAL SYSTEM OF AN ENDOSCOPE, ENDOSCOPE, STEREO-VIDEO ENDOSCOPE, AND METHOD FOR PRODUCING AN OPTICAL SYSTEM

(71) Applicant: OLYMPUS WINTER & IBE GMBH, Hamburg (DE)

(72) Inventor: Jianxin ZHAO, Hamburg (DE)

(73) Assignee: OLYMPUS WINTER & IBE GMBH, Hamburg (DE)

(21) Appl. No.: 16/986,445

(22) Filed: Aug. 6, 2020

Related U.S. Application Data
(63) Continuation of application No. PCT/EP2019/052515, filed on Feb. 1, 2019.

(30) Foreign Application Priority Data
Feb. 6, 2018 (DE) 102018102641.2

(51) Int. Cl. G02B 23/24; G02B 1/10; G02B 17/04

(52) U.S. Cl. CPC G02B 23/2415; G02B 23/243; A61B 1/00193; G02B 17/04; G02B 1/10

(57) ABSTRACT

An optical system of an endoscope having a lateral viewing direction, the optical system including: a sideways looking distal optical assembly; and a proximal optical assembly, wherein light bundles incident from an object space are guided along a beam path by the distal and proximal optical assemblies and imaged on an image sensor; the distal optical assembly comprises a deflection prism assembly having first and second prisms arranged in a direction of incident light; the first prism has first inlet and inclined first outlet sides; the second prism has second inlet, reflection, and second outlet sides; and the first outlet side of the first prism and/or the second inlet side of the second prism have a coating in a region outside of the beam path to form an air gap in a region of the beam path between the first outlet and second inlet sides.

### CROSS-REFERENCE TO RELATED APPLICATION

The present application is a continuation of PCT/EP2019/052515 filed on Feb. 1, 2019, which is based upon and claims the benefit to DE 10 2018 102 641.2 filed on Feb. 6, 2019, the entire contents of each of which are incorporated herein by reference.

### BACKGROUND

### Field

The present disclosure relates to an optical system of an endoscope having a lateral viewing direction, comprising a sideways looking distal optical assembly and a proximal optical assembly, wherein light bundles incident from an object space are guided along a beam path by the optical assemblies and imaged on an image sensor, wherein the distal optical assembly comprises a deflection prism assembly which comprises a first prism and a second prism which follow one another in the direction of incident light, wherein the first prism comprises a first inlet side and a first outlet side inclined with respect thereto, and wherein the second prism comprises a second inlet side, a reflection side and a second outlet side. The present disclosure additionally relates to an endoscope having a lateral viewing direction as well as a stereo-video endoscope having a lateral viewing direction. Finally, the present disclosure relates to a method for producing an optical system of an endoscope having a lateral viewing direction, comprising a sideways looking distal optical assembly and a proximal optical assembly, wherein light bundles incident from an object space are guided along a beam path by the optical assemblies and imaged on an image sensor, wherein the distal optical assembly comprises a deflection prism assembly which comprises a first prism and a second prism which follow one another in the direction of incident light, wherein the first prism comprises a first inlet side and a first outlet side inclined with respect thereto, and wherein the second prism comprises a second inlet side, a reflection side and a second outlet side.

### Prior Art

Endoscopes, for example video endoscopes, further for example stereo-video endoscopes, comprise an optical system, with which light bundles entering at a distal tip of an endoscope shaft are diverted to one or more image sensors. In the case of endoscopes having a lateral viewing direction, the viewing direction of the optical system differs from a longitudinal axial direction of the endoscope shaft and encloses with the latter a predefined or variable angle of typically between 25° and 70°. Therefore, both endoscopes having a fixed lateral viewing direction and endoscopes having a variable lateral viewing direction, which are also referred to as V-DOV endoscopes, are known.

In the case of stereo-video endoscopes, a stereoscopic pair of images and/or two stereoscopic video channels are acquired with the aid of the optical system. With such instruments, it is possible to produce a 3D image of an object located distally before the end of the endoscope shaft in an examination and/or operating space.

The optical system of such endoscopes comprises a deflection prism assembly made of multiple prisms which repeatedly reflect the beams of light entering the optical system from an object space at a specific angle to the longitudinal axis of the endoscope shaft and deflect them in an unreversed way in the direction of a left and of a right lens system channel. The lens system channels each comprise an image sensor for capturing the image data.

A deflection prism assembly of an endoscope is, for example, known from EP 2 593 827 B1.

The deflection prism assembly of a stereo-video endoscope typically comprises two prisms which are cemented to one another at their common interface. The incident light bundles are reflected off two reflecting interfaces of the second prism, which are located diagonally both to the optical axis of the inlet lens and to the longitudinal axis of the endoscope shaft. The second prism is situated in the direction of incident light behind the first prism which is arranged immediately behind an inlet lens. The last reflection, before the beams of light subsequently enter a proximal optical assembly which can consist of one or more lens system channels, typically takes place under total reflection. In other words, this last reflection therefore takes place at a glass-air interface.

### SUMMARY

An object is to provide an optical system of an endoscope having a lateral viewing direction, an endoscope having a lateral viewing direction, a stereo-video endoscope having a lateral viewing direction, as well as a method for producing an optical system of an endoscope having a lateral viewing direction, wherein a deflection prism assembly of the optical system is to have a simplified configuration.

Such object can be solved by an optical system of an endoscope having a lateral viewing direction, comprising a sideways looking distal optical assembly and a proximal optical assembly, wherein light bundles incident from an object space are guided along a beam path by the optical assemblies and imaged on an image sensor, wherein the distal optical assembly comprises a deflection prism assembly which comprises a first prism and a second prism which follow one another in the direction of incident light, wherein the first prism comprises a first inlet side and a first outlet side inclined with respect thereto, and wherein the second prism comprises a second inlet side, a reflection side and a second outlet side, wherein the optical system is further developed in that the first outlet side of the first prism and/or the second inlet side of the second prism is/are provided with a coating in a region outside of the beam path, and an air gap is provided in the region of the beam path between the first outlet side and the second inlet side.

The configuration of the optical system can be based on the following considerations. Total reflection takes place at a second inlet side of the second prism of the deflection prism assembly before the light bundles are coupled into the proximal optical assembly, for example a left and a right lens system channel. For the required total reflection, a jump in the refractive index can be required between the material of the second prism and a joining medium at the corresponding interface. A glass-air interface is well suited to total reflection.

The air gap can be produced according to the prior art in that a mask can be arranged between the two prisms so that the air gap is provided between the first outlet side and the second inlet side. At the same time, the installation space of the optical system is to be as small as possible. The mask can therefore be configured with the smallest possible material thickness. Producing a very thin mask is, however, a technically elaborate process. Assembling the optical system is also a technical challenge since such a thin mask can only be handled with the utmost care and requires a great deal of experience. It is difficult to position the mask correctly during assembly and, above all, not to damage it.

The total reflection which takes place at the second inlet side merely calls for a part of this side of the prism. In other words, the jump in the refractive index, which is provided by the glass-air material transition, does not have to be realized all-over. It is sufficient if this jump is present in a region of the beam path. The mask is situated outside of the beam path, that is to say at locations where no total reflection takes place, according to the prior art.

Regions of the first outlet side and of the second inlet side inside and outside of the beam path of the optical system can be defined as follows within the context of the present description: those bundles of rays, which are imaged by the optical system on a light-sensitive surface of an image sensor, run along the beam path.

The optical system can comprise at least one image sensor, wherein light bundles incident from a field of view located in an object space are imaged by the distal and the proximal optical assembly on the light-sensitive surface of the at least one image sensor.

The light bundles running in the beam path penetrate the interfaces of the optical components provided in the optical system. That region of such an interface which is penetrated by the light bundles is regarded as a region located in the beam path. Those regions of the surfaces, which are not penetrated by the bundles of rays, are accordingly located outside of the beam path. The border between these two regions is defined by those bundles of rays which enter the optical system from the edge of the field of view and are still imaged on the at least one image sensor.

Instead of a mask, a coating can be provided in order to produce the desired air gap between the first outlet side and the second inlet side. Optionally, the coating can be applied to the first outlet side of the first prism or to the second inlet side of the second prism in a region outside of the beam path. It is likewise provided that a coating can be applied both to the first outer side of the first prism and to the second inlet side of the second prism. With the aid of the coating it is ensured that the air gap is provided between the first outlet side and the second inlet side and total reflection takes place at the second inlet side.

The optical system can have a simplified configuration since it is possible to dispense with the fragile and extremely thin mask. This simplifies the production and the assembly of the optical system. The coating provided can, for example, be applied with the aid of a suitable thin-layer method such as vapor deposition or sputter deposition. Such a production step can be controlled well and reliably, technically, especially since the region to be coated does not have to be observed with the greatest precision. The desired technical functionality, namely the production of the air gap, is also guaranteed, if the coating is not applied exactly at the provided position. The only crucial factor is that the coating is present in a region outside of the beam path. It is therefore provided that the region of the first outlet side and/or of the second inlet side located outside of the beam path can be merely coated in certain regions.

The optical system can be further developed in that the coating can be provided exclusively on the first outlet side of the first prism or on the second inlet side of the second prism, such as exclusively on the first outlet side of the first prism. The fact that the coating is solely, optionally, provided on the first outlet side of the first prism or on the second inlet side of the second prism means that it is not necessary to coat two surfaces. Affixing the coating to the first outlet side of the first prism has proven to be particularly efficient in practice.

According to a further embodiment, it is provided that the coating can have a layer thickness between 2 μm and 20 μm, such as between 5 μm and 15 μm, or between 5 μm and 10 μm. In order to attain the effect of total reflection, a thin air gap can be sufficient. In addition, installation space can be saved by a thin air gap. A thin air gap can be realized by deploying a coating. The size of the air gap lies in a range which cannot be achieved when an aperture is used. A size of the air gap, which lies in one of the indicated intervals, has proven to be particularly practicable.

The optical system can be further developed in that the coating is a metal coating, such as a chromium coating or a coating containing, at least for the most part, chromium. A metal coating shows good adhesion to glass surfaces. In addition, metal is ductile, so that it is unlikely that the coating will chip. A chromium coating has proven to be particularly suitable.

The deposition of the coating can only require a little adaptation or no adaptation at all of the production process of the optical system. Thus, the prisms can gradually be bonded to one another by a pressing force, for example with the aid of a punch, directly in the prism holder. Such process can be similar or identical to the conventional mask bonding process. The corresponding adhesive connections can be provided in the region of the coating.

According to a further embodiment, the optical system can be further developed in that the proximal optical assembly comprises a left lens system channel and a right lens system channel which have a similar configuration, and the distal optical assembly can be configured to couple light bundles incident from the object space into the left lens system channel and into the right lens system channel, and wherein the distal optical assembly comprises an inlet lens, the deflection prism assembly and an outlet lens which follow one another in the direction of incident light.

The optical system according to this embodiment can be suitable for deployment in a stereo-video endoscope. The use in a stereo-video endoscope is particularly useful, since the reflecting surfaces of the deflection prism assembly are comparatively large with this type of endoscope. Accordingly, a comparatively large, but extremely thin mask can be used according to the prior art in order to produce the required air gap as well. The handling of such a mask is very demanding and is not successful in all cases. This leads to delays in production and unnecessary rejects. These technical disadvantages can be avoided with the disclosed optical system.

Such object can also be solved by a stereo-video endoscope having a lateral viewing direction, which is further developed in that the latter comprises an optical system according to the above embodiment.

Additionally, such object can be solved by an endoscope having a lateral viewing direction, which is further developed in that the latter comprises an optical system according to one or more of the embodiments indicated above.

The endoscope or the stereo-video endoscope can have, for example, a variable lateral viewing direction.

The endoscope and the stereo-video endoscope have the same or similar advantages to those which have already been mentioned above with respect to the optical system, so a repetition shall be dispensed with.

Such object can also be solved by a method for producing an optical system of an endoscope having a lateral viewing direction, comprising a sideways looking distal optical assembly and a proximal optical assembly, wherein light bundles incident from an object space are guided along a beam path by the optical assemblies and imaged on an image sensor, wherein the distal optical assembly comprises a deflection prism assembly which comprises a first prism and a second prism which follow one another in the direction of incident light, wherein the first prism comprises a first inlet side and a first outlet side inclined with respect thereto, and wherein the second prism comprises a second inlet side, a reflection side and a second outlet side, wherein the method can be further developed in that the first outlet side of the first prism and/or the second inlet side of the second prism is/are provided with a coating in a region outside of the beam path, and the prisms are arranged in such a manner that an air gap is provided in the region of the beam path between the first outlet side and the second inlet side.

The method for producing the optical system is simpler and feasible. It is no longer necessary to handle a fragile and extremely thin mask which is easy to damage. As a result, the production of the optical system is more rapid, wherein better results are attained more reliably.

Incidentally, the method for producing the optical system has the same or similar advantages to those which have already been mentioned above with respect to the optical system itself.

The method can be further developed in that the coating can be exclusively applied to the first outlet side of the first prism or to the second inlet side of the second prism, such as exclusively to the first outlet side of the first prism.

The method can be additionally further developed in that the coating can be applied with a layer thickness between 2 μm and 20 μm, such as between 5 μm and 15 μm or between 5 μm and 10 μm.

### BRIEF DESCRIPTION OF THE DRAWINGS

Further features will become apparent from the description of embodiments, together with the claims and the appended drawings. Embodiments can fulfil individual features or a combination of multiple features.

The embodiments are described below without limiting the general concept of the invention by means of exemplary embodiments with reference to the drawings, wherein reference is expressly made to the drawings regarding all of the details which are not explained in greater detail in the text, wherein:

FIG. 1 illustrates a schematically simplified perspective view of an endoscope having a lateral viewing direction,

FIG. 2 illustrates a schematically simplified representation of an optical system of a stereo endoscope,

FIG. 3 illustrates a further schematically simplified sectional view of an optical system of an endoscope having a lateral viewing direction,

FIG. 4 illustrates a schematically simplified top view of a second inlet side of a second prism of a deflection prism assembly of an optical system, wherein the second inlet side is provided with a coating in a region outside of the beam path.

In the drawings, the same or similar elements and/or parts are, in each case, provided with the same reference numerals so that they are not introduced again in each case.

### DETAILED DESCRIPTION

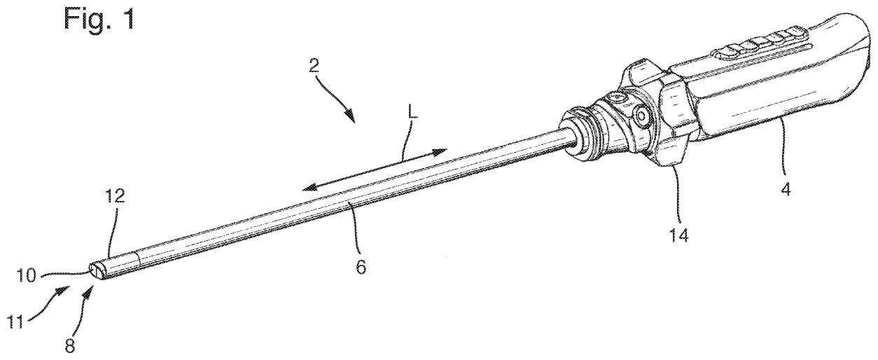

FIG. 1 shows a schematically simplified perspective representation of an endoscope 2, comprising a proximal handle 4, to which a rigid endoscope shaft 6, merely by way of example, is joined. The endoscope shaft 6 can likewise have a flexible or semi-flexible configuration. At the distal tip 8 of the endoscope shaft 6 there is situated an inlet window 10, through which light from an object space 11, for example an operating or observation field, enters an optical system of the endoscope 2 which is not visible in FIG. 1. The optical system is, for example, arranged in a distal portion 12 of the endoscope shaft 6. The optical system images objects, which are situated in the object space 11, on at least one image sensor. For this purpose, the optical system comprises at least one image sensor. The image sensor(s) can be one(s) having a high resolution, e.g. HD, 4K or following technologies.

The endoscope 2 is, for example, a surgical instrument. It is an endoscope 2 having a fixed lateral viewing direction or having a variable lateral viewing direction. By way of example, an endoscope 2 having a fixed lateral viewing direction is depicted, the inlet window 10 of which is assembled, inclined, in the endoscope shaft 6, so that an optical axis of an inlet lens of the optical system having a longitudinal extension direction L of the endoscope shaft 6, which is not depicted in FIG. 1, encloses a fixed angle which is, for example, between 25° and 70°.

An alteration of the viewing direction about the longitudinal axis of the endoscope shaft 6 is, for example, caused by a rotation of the handle 4. The optical system provided in the distal portion 12 also rotates during such a rotation of the handle 4. In order to retain the horizontal position of the displayed image, a rotating wheel 14 is secured during a rotation of the handle 4, as a result of which the image sensors in the interior of the endoscope shaft 6 do not execute the rotating movement as well.

The depicted endoscope 2 can be a video endoscope, such as a stereo-video endoscope.

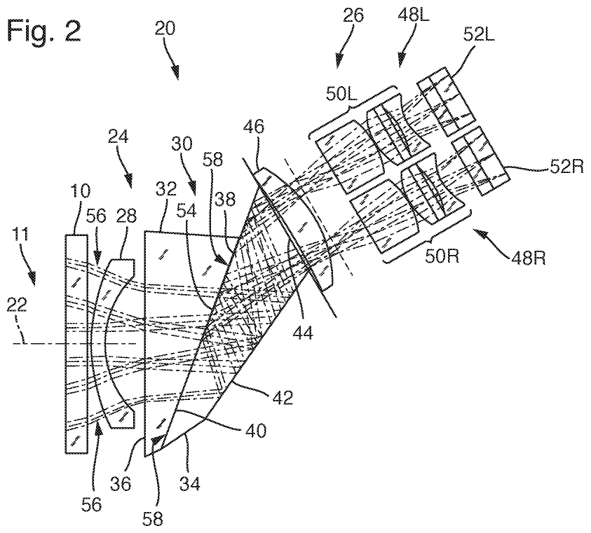

FIG. 2 shows an optical system 20, as it is deployed in a stereo endoscope, such as a stereo-video endoscope. The optical system 20 defines a fixed lateral viewing direction. The optical axis 22, which is the optical axis of an inlet lens 28 of the optical system 20, encloses a fixed angle of, for example, 30° with the longitudinal extension direction L of the endoscope shaft 6. The optical system 20 comprises a sideways looking distal optical assembly 24 and a proximal optical assembly 26. Since the depicted optical system 20 is the system of a stereo-video endoscope, the proximal optical assembly 26 comprises a left lens system channel 48L and a right lens system channel 48R. The two lens system channels 48L, 48R have a similar configuration. The distal optical assembly 24 is configured to couple light bundles incident from the object space 11 both into the left lens system channel 48L and into the right lens system channel 48R of the proximal optical assembly 26.

The light bundles incident from the object space 11 through the inlet window 10 (depicted as a dotdashed line) first strike the inlet lens 28 of the distal optical assembly 24 and, subsequently, travel into a deflection prism assembly 30. Viewed in the direction of incident light, the deflection prism assembly 30 comprises a first prism 32 and a following second prism 34.

In the direction of incident light, the light bundles, which leave the inlet lens 28, first pass through a first inlet side 36 of the first prism 32. The light bundles pass through the body of the first prism 32 and travel to the first outlet side 38 thereof. The first outlet side 38 is inclined with respect to the first inlet side 36. Adjacent to the first outlet side 38 there is situated an air gap 54 which is not depicted in FIG. 2 due to its small dimensions. The light bundles pass through the air gap 54 and enter the second prism 34 via a second inlet side 40. The second prism 34 comprises a reflection side 42 which is inclined with respect to the second inlet side 40. The light bundles are reflected by the reflection side 42 of the second prism 34. From there, they travel from the rear side to the second inlet side 40 of the second prism 34.

An interface between the material of the second prism 34 and the air gap 54 is present on the second inlet side 44, so that a jump in the refractive index is provided on the second inlet side 44. The light bundles, coming from internally, are therefore totally reflected by the second inlet side 40 of the second prism 34 and, subsequently, leave the second prism 34 at its second outlet side 44. From there, the light bundles continue traveling in the direction of incident light to an outlet lens 46 of the distal optical assembly 24.

The proximal optical assembly 26 comprises the left lens system channel 48L and the right lens system channel 48R. The two lens system channels 48L, 48R have a similar or identical configuration and are additionally aligned with one another so that their respective optical axes are located parallel to one another. The left lens system channel 48L comprises the imaging left lens group SOL which images the incident light on the left image sensor 52L. The right lens system channel 48R comprises the imaging right lens group 50R which images the incident light bundles on the right image sensor 52R.

The light bundles running through the optical system 20 define a beam path inside of the optical system 20. The boundaries of the beam path are fixed by the borderline light bundles 56. The borderline light bundles 56 are those light bundles which are incident from the object space 11 and are still imaged on a light-sensitive surface of the image sensors 52L, 52R. The light bundles penetrate the individual interfaces of the optical components of the optical system 20. Those regions, which are penetrated by the light bundles, are regarded as regions inside of the beam path. The regions which are located outside, that is to say are no longer penetrated by light bundles, are regarded as being located outside of the beam path. The borderline light bundles 56 define the boundary between these two regions.

In order to produce the air gap 54 between the first outlet side 38 and the second inlet side 40, the first outlet side 38 of the first prism 32 and/or the second inlet side 40 of the second prism 34 is/are provided with a coating in a region outside of the beam path which is denoted, by way of example, with reference numeral 58 in FIG. 2. The coating has a layer thickness between 2 μm and 20 μm and ensures that an air gap 54 is provided between the first outlet side 38 and the second inlet side 40. It is likewise provided that the coating is provided exclusively on the first outlet side 38 of the first prism 32 or exclusively on the second inlet side 40 of the second prism 34. The coating can be a metal coating, for example a chromium coating or a coating containing chromium for the most part.

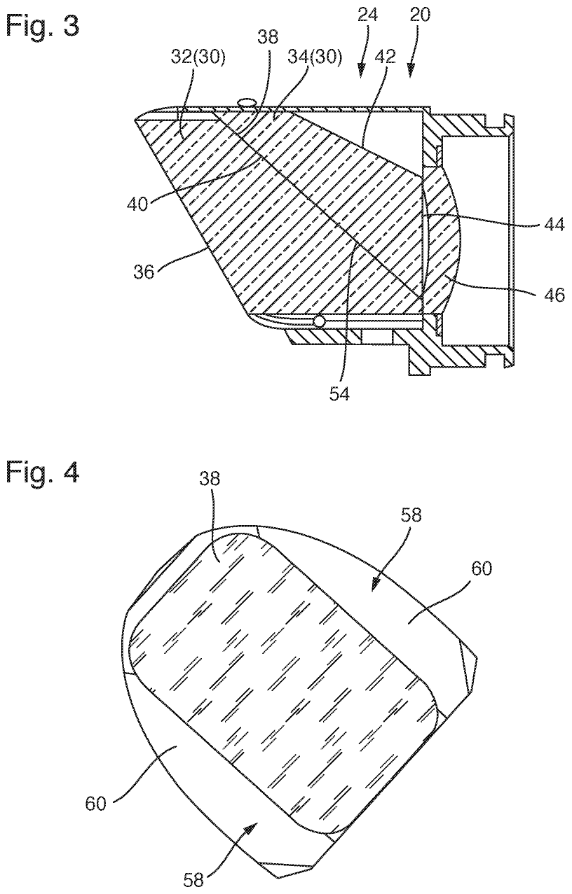

FIG. 3 shows a part of a further optical system 20 of an endoscope 2. The distal optical assembly 24 is depicted, wherein an inlet lens which may be provided is not depicted. The optical system shown is deployed for image or video endoscopes which do not supply stereo images. The optical system comprises a deflection prism assembly 30 having a first prism 32 and a second prism 34.

Light bundles incident from an object space pass through the deflection prism assembly 30. They enter the body of the first prism 32 through the first inlet side 36 of the first prism 32 and leave the body at the first outlet side 38. The beams of light pass through an air gap 54 between the first outlet side 38 and the second inlet side 40 of the second prism 34. In the second prism 34, the light bundles are reflected by the reflection side 42 of the second prism 34 and travel from the rear side to the second inlet side 40. There, they are totally reflected, since the air gap 54 is provided between the second inlet side 40 and the first outlet side 38. The light bundles leave the deflection prism assembly 30 via the second outlet side 44 of the second prism 34. They travel via the outlet lens 46 into a proximal optical assembly of the optical system 20, which is not depicted in FIG. 3, which proximal optical assembly finally directs the light bundles to an image sensor.

FIG. 4 shows a top view of the first outlet side 38 of the first prism 32. In a region 58 which is located outside of the beam path, the first outlet side 38 is provided with the coating 60. The top view depicted in FIG. 4 also applies similarly to the first prism 32 of the optical system depicted in FIG. 2.

The coating 60 is, for example, applied with a thin-layer method to the first outlet side 38 of the first prism 32.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMERALS

2 Endoscope
4 Handle
6 Endoscope shaft
8 Distal tip
10 Inlet window
11 Object space
12 Distal portion
14 Rotating wheel
20 Optical system
22 Optical axis
24 Distal optical assembly
26 Proximal optical assembly
28 Inlet lens
30 Deflection prism assembly
32 First prism
34 Second prism
36 First inlet side
38 First outlet side
40 Second inlet side
42 Reflection side
44 Second outlet side
46 Outlet lens
48L Left lens system channel
48R Right lens system channel
50L Left lens group
50R Right lens group
52L Left image sensor
52R Right image sensor
54 Air gap
56 Borderline light bundles
58 Region
60 Coating
L Longitudinal extension direction

What is claimed is:

1. An optical system of an endoscope having a lateral viewing direction, the optical system comprising:
a sideways looking distal optical assembly; and
a proximal optical assembly;
wherein light bundles incident from an object space are guided along a beam path by the distal optical assembly and the proximal optical assembly and imaged on an image sensor;
the distal optical assembly comprises a deflection prism assembly which comprises a first prism and a second prism which follow one another in a direction of incident light;
the first prism comprises a first inlet side and a first outlet side inclined with respect to the first inlet side;
the second prism comprises a second inlet side, a reflection side and a second outlet side; and
one or more of the first outlet side of the first prism and the second inlet side of the second prism are provided with a coating in a region outside of the beam path to form an air gap in a region of the beam path between the first outlet side and the second inlet side.

2. The optical system according to claim 1, wherein the coating is provided only on one of the first outlet side of the first prism or on the second inlet side of the second prism.

3. The optical system according to claim 2, wherein the coating is only provided on the first outlet side of the first prism.

4. The optical system according to claim 1, wherein the coating has a layer thickness between 2 μm and 20 μm.

5. The optical system according to claim 4, wherein the layer thickness is between 5 μm and 15 μm.

6. The optical system according to claim 4, wherein the layer thickness is between 5 μm and 10 μm.

7. The optical system according to claim 1, wherein the coating is a metal coating.

8. The optical system according to claim 4, wherein the metal coating is one of a chromium coating or a coating at least partially containing chromium.

9. The optical system according to claim 1, wherein:
the proximal optical assembly comprises a left lens system channel and a right lens system channel, and the distal optical assembly is configured to couple light bundles incident from the object space into the left lens system channel and into the right lens system channel, and
the distal optical assembly comprises an inlet lens, the deflection prism assembly and an outlet lens which follow one another in the direction of incident light.

10. An endoscope having a lateral viewing direction, the endoscope comprising the optical system according to claim 1.

11. A stereo-video endoscope having a lateral viewing direction, the stereo-video endoscope comprising the optical system according to claim 9.

12. A method for producing an optical system of an endoscope having a lateral viewing direction, the endoscope comprising a sideways looking distal optical assembly and a proximal optical assembly, wherein light bundles incident from an object space are guided along a beam path by the distal optical assembly and the proximal optical assembly and imaged on an image sensor, wherein the distal optical assembly comprises a deflection prism assembly which comprises a first prism and a second prism which follow one another in a direction of incident light, wherein the first prism comprises a first inlet side and a first outlet side inclined with respect thereto, and the second prism comprises a second inlet side, a reflection side and a second outlet side, the method comprising coating one or more of the first outlet side of the first prism and the second inlet side of the second prism in a region outside of the beam path to form an air gap in the region of the beam path between the first outlet side and the second inlet side.

13. The method according to claim 12, wherein the coating is applied only to one of the first outlet side of the first prism or to the second inlet side of the second prism.

14. The method according to claim 13, wherein the coating is applied only to the first outlet side of the first prism.

15. The method according to claim 12, wherein the coating is applied with a layer thickness between 2 μm and 20 μm.

16. The method according to claim 15, wherein the layer thickness is between 5 μm and 15 μm.

17. The method according to claim 15, wherein the layer thickness is between 5 μm and 10 μm.
